SEQUENCE LISTING (4 sequences)

SEQ ID NO: 1 - RNA, 22 nt, synthetic construct
  source 1..22: mol_type = other RNA
  misc_feature 1..22: note = miR-15a guide strand
  modified_base 1: mod_base = OTHER; note = uracil or 5-fluorouracil
  misc_feature 4: note = cytidine or cytosine arabinoside
  misc_feature 7: note = cytidine or cytosine arabinoside
  modified_base 11: mod_base = OTHER; note = uracil or 5-fluorouracil
  modified_base 14: mod_base = OTHER; note = uracil or 5-fluorouracil
  modified_base 17..19: mod_base = OTHER; note = uracil or 5-fluorouracil
  modified_base 21: mod_base = OTHER; note = uracil or 5-fluorouracil
  misc_feature 9: note = cytidine or cytosine arabinoside
tagcagcacataatggtttgtg

SEQ ID NO: 2 - RNA, 22 nt, synthetic construct
  source 1..22: mol_type = other RNA
  misc_feature 1..22: note = miR-15a passenger strand
  misc_feature 1: note = cytidine or cytosine arabinoside
  misc_feature 5..6: note = cytidine or cytosine arabinoside
  misc_feature 15: note = cytidine or cytosine arabinoside
  misc_feature 18..19: note = cytidine or cytosine arabinoside
  misc_feature 21: note = cytidine or cytosine arabinoside
caggccatattgtgctgcctca

SEQ ID NO: 3 - RNA, 23 nt, Homo sapiens
  source 1..23: mol_type = mRNA
  3'UTR 1..23: gene = Wee1 mRNA nucleotides 2455-2477
ttcgtcgtgtggtggtgtcacca

SEQ ID NO: 4 - RNA, 23 nt, Homo sapiens
  source 1..23: mol_type = mRNA
  3'UTR 1..23: gene = Wee1 mRNA nucleotides 2734-2756
atcgtcgtttaagtgttccgtta

(19) United States
(12) Patent Application Publication
(10) Pub. No.: US 2024/0011038 A1
(43) Pub. Date: Jan. 11, 2024

(54) CYTARABINE-MODIFIED miRNA FOR TREATING CANCER

(71) Applicant: Curamir Therapeutics, Inc., Woburn, MA (US)

(72) Inventor: Andrew Fesler, Woburn, MA (US)

(21) Appl. No.: 18/338,089

(22) Filed: Jun. 20, 2023

Related U.S. Application Data
(60) Provisional application No. 63/354,009, filed on Jun. 21, 2022.

(51) Int. Cl. C12N 15/113; A61P 35/02

(52) U.S. Cl. CPC C12N 15/1135; A61P 35/02; C12N 2310/141; C12N 2310/334; C12N 2310/335; C12N 2310/343

(57) ABSTRACT

A composition that includes a micro-RNA mimic containing one or more cytosine nucleosides in which at least one cytosine nucleoside is replaced by cytosine arabinoside. Also provided is a method for killing a cancer cell by contacting it with a composition that includes a micro-RNA mimic containing cytosine arabinoside and 5-fluorouracil in which the micro-RNA mimic is miR-15a, miR-16, miR-129, miR-194, miR-192, miR-139, miR-140, or miR-145. Further, disclosed is a method for treating cancer by administering a composition containing a micro-RNA mimic having a guide strand and a passenger strand in which at least one cytosine nucleoside in the guide strand or the passenger strand has been replaced by cytosine arabinoside.

### CROSS-REFERENCE TO RELATED APPLICATIONS

The present application claims the benefit of U.S. Provisional Patent Application Ser. No. 63/354,009, filed on Jun. 21, 2022. The content of the prior application is hereby incorporated by reference in its entirety.

### SEQUENCE LISTING XML

A computer readable file containing a sequence listing is being electronically co-filed herewith via EFS-Web. The computer readable file, submitted under 37 CFR § 1.831(a) and having the file name “41D9811.XML” was created on Jun. 15, 2023 and has a size of 11,884 bytes. The content of the computer readable file is hereby incorporated by reference in its entirety.

### BACKGROUND

Acute myeloid leukemia (“AML”) presents a significant clinical challenge. AML cases, incidence, and deaths have been rising worldwide over the past 30 years and clinical outcomes remain poor. AML is most prevalent in elderly populations, with greater than 75% of cases occurring in patients over 65 years old. The 5-year overall survival rate for these patients is approximately 24% with median survival of 8.5 months. Traditional chemotherapeutic approaches based on anthracycline and cytarabine are somewhat effective for treating AML. However, elderly patients are not candidates for intensive chemotherapy.

Cytarabine (cytosine arabinoside or “ara-C”) is an antimetabolic agent that combines a cytosine base with an arabinose sugar. Cytarabine inhibits DNA synthesis in dividing cells resulting in cell death. It has been used as a chemotherapeutic for the treatment of various leukemias, e.g., AML, as well as lymphoma.

Aside from chemotherapy, cancer treatments based on micro-RNA (“miRNA”) are being developed. miRNAs are short non-coding RNAs with important roles in regulating gene expression. Individual miRNAs can inhibit the expression of many different target genes through imperfect base pairing to their 3′ untranslated region.

The importance of miRNAs in cancer was first identified in chronic lymphocytic leukemia, in which miRNA 15a (“miR-15a”) and miRNA 16 (“miR-16”) were found to have reduced expression. Since that discovery, miRNAs have been shown to play a significant role in many different cancer types.

There remains a need to develop new therapeutic approaches having reduced toxicity with the goal of extending survival for patients suffering from AML and other leukemias.

### SUMMARY

To meet the above need, a composition is provided that includes a miRNA mimic containing one or more cytosine nucleosides in which at least one cytosine nucleoside is replaced by cytosine arabinoside (“ara-C”).

Also provided is a method for killing a cancer cell by contacting it with a composition that includes a miRNA mimic containing ara-C and 5-fluorouracil (“5-FU”) in which the miRNA mimic is miR-15a, miR-16, miR-129, miR-194, miR-192, miR-139, miR-140, or miR-145.

Further, disclosed is a method for treating cancer by administering to a subject an effective amount of a composition containing a miRNA mimic having a guide strand and a passenger strand that each contains one or more cytosine nucleosides in which at least one cytosine nucleoside in the guide strand or at least one cytosine nucleoside in the passenger strand is replaced by ara-C.

The details of one or more embodiments of the invention are set forth in the description below. Other features, objects, and advantages of the invention will be apparent from the description, from the drawings, and from the claims.

### BRIEF DESCRIPTION OF THE DRAWINGS

The description below refers to the accompanying drawings, of which:

FIG. 1A is a modified miRNA mimic in which cytosine nucleosides in the guide strand (underlined) have been replaced with ara-C. G=guide strand (SEQ ID NO: 1); P=passenger strand (SEQ ID NO: 2).

FIG. 1B is a modified miRNA mimic in which cytosine nucleosides in the guide strand (underlined) have been replaced with ara-C and uracil bases (double underlined) in the guide strand have been replaced with 5-FU. G=guide strand (SEQ ID NO: 1); P=passenger strand (SEQ ID NO: 2).

FIG. 1C is a modified miRNA mimic in which cytosine nucleosides in the passenger strand (underlined) have been replaced with ara-C. G=guide strand (SEQ ID NO: 1); P=passenger strand (SEQ ID NO: 2).

FIG. 1D is a modified miRNA mimic in which cytosine nucleosides in the passenger strand (underlined) have been replaced with ara-C and uracil bases (double underlined) in the guide strand have been replaced with 5-FU. G=guide strand (SEQ ID NO: 1); P=passenger strand (SEQ ID NO: 2).

FIG. 1E is a modified miRNA mimic in which cytosine nucleosides in the passenger strand and the guide strand (underlined) have been replaced with ara-C and uracil bases (double underlined) in the guide strand have been replaced with 5-FU. G=guide strand (SEQ ID NO: 1); P=passenger strand (SEQ ID NO: 2).

FIG. 1F is a modified miRNA mimic in which uracil bases (double underlined) in the guide strand have been replaced with 5-FU. G=guide strand (SEQ ID NO: 1); P=passenger strand (SEQ ID NO: 2).

FIG. 2 is a plot of optical density (“OD”) versus days post-transfection by lipofection of the indicated miRNAs into MV-4-11 acute myeloid leukemia cells. Negative=negative control miRNA, 5-FU miR-15a=miR-15a mimic with 5-fluorouracil (“5-FU”) in the guide strand, ara-C 5-FU miR-15a=miR-15a mimic with 5-FU and cytosine arabinoside (“ara-C”) in the guide strand, Passenger ara-C 5-FU miR-=miR-15a mimic with ara-C in the passenger strand and 5-FU in the guide strand, Passenger ara-C miR-15a=miR-15a mimic with ara-C in the passenger strand, ara-C miR-15a=miR-15a mimic with ara-C in the guide strand.

FIG. 3 is a plot of percent cell viability versus concentration of the indicated miRNAs measured 6 days after vehicle-free transfection into MV-4-11 cells. miRNA definitions are as described in the legend to FIG. 2.

FIG. 4 is a plot of OD versus days post-transfection by lipofection of the indicated miRNAs into REH acute lymphocytic leukemia (“ALL”) cells. miRNAs are defined above.

FIG. 5 is a plot of percent cell viability versus concentration of the indicated miRNAs measured 6 days after vehicle-free transfection into REH cells. miRNAs are as described above.

FIG. 6A shows an alignment between the guide strand of miR-15a and a segment of the 3′ untranslated region (“UTR”; nucleotides 2455-2477 of Genbank NM_003390.4) of the Wee1 gene (SEQ ID NO: 3).

FIG. 6B shows an alignment between the guide strand of miR-15a and another segment of the 3′ UTR (nucleotides 2734-2756 of Genbank NM_003390.4) of the Wee1 gene (SEQ ID NO: 4).

FIG. 7 is a bar graph showing relative quantity (“RQ”) of Wee1 gene mRNA measured by quantitative reverse-transcription PCR (“qRT-PCR”) in MV-4-11 cells transfected with the indicated miRNAs. The miRNAs are defined above in the legend to FIG. 2. *=significantly different from negative control p<0.05 by T-test.

### DETAILED DESCRIPTION

As set out in the SUMMARY section above, a composition is disclosed that includes a miRNA mimic containing one or more cytosine nucleosides in which at least one cytosine nucleoside is replaced by ara-C. In the disclosed composition, the miRNA mimic can include a guide strand and a passenger strand. The ara-C can be present in the in the guide strand, in the passenger strand, or in both strands.

The composition can include a pharmaceutically acceptable excipient or carrier. The excipient can be a cationic polymer such as polyethyleneimine, and the carrier can be a lipid carrier, e.g., or a lipid nanoparticle. Additional examples of excipients and carriers are set forth in Ju et al., US Patent Application Publication 2019/0062754 (“Ju”), the contents of which are hereby incorporated by reference in its entirety.

In certain compositions, a single cytosine nucleoside in the guide strand, in the passenger strand, or in both strands is replaced by ara-C. In a particular composition, all of the cytosine nucleosides in the passenger strand are replaced with ara-C. In another composition, all of the cytosine nucleosides in the guide strand are replaced with cytosine arabinoside. Also within the scope of the invention is a composition in which all cytosine nucleosides in both the passenger strand and the guide strand are replaced by ara-C.

The compositions described above can include a guide strand that contains one or more uracil bases in which at least one uracil base is replaced by 5-halouracil. The 5-halouracil can be 5-bromouracil, 5-chlorouracil, 5-iodouracil, or 5-fluorouracil. In a particular composition the 5-halouracil is 5-fluorouracil (“5-FU”).

In certain compositions, a single uracil base in the guide strand is replaced with 5-FU. In other compositions, all of the uracil bases in the guide strand are replaced with 5-fluoruracil.

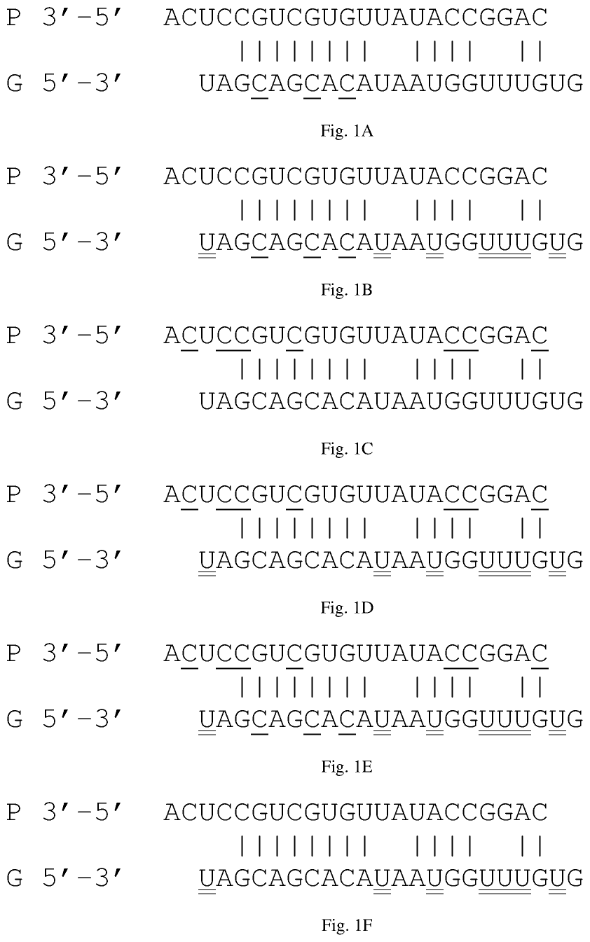

Any combination of ara-C- and 5-FU-modified guide strands and passenger strands can be included in the composition. See FIGS. 1A-1F. For example, one composition contains a guide strand and a passenger strand both having all cytosine nucleosides replaced by ara-C, together with all uracil bases in the guide strand replaced by 5-FU. In another composition, all of the cytosine nucleosides in the passenger strand are replaced by ara-C, none of the cytosine nucleosides in the guide strand are modified, and all of the uracil bases in the guide strand are replaced by 5-FU. Additional compositions can include (i) an unmodified passenger strand together with a guide strand having all cytosine nucleosides replaced by ara-C, and (ii) an unmodified guide strand and a passenger strand having all cytosines replaced by ara-C.

In particular compositions, the miRNA mimic can be miR-15a, miR-16, miR-129, miR-194, miR-192, miR-139, miR-140, or miR-145. Each of these miRNA mimics can include a guide strand and a passenger strand modified with ara-C, 5-FU, or both as set forth, supra.

In an exemplary composition, the miRNA mimic is a miR-15a mimic that includes a guide strand consisting of the sequence UAGCAGCACAUAAUGGUUUGUG (SEQ ID NO: 1) and a passenger strand consisting of the sequence CAGGCCAUAUUGUGCUGCCUCA (SEQ ID NO: 2). In a specific composition, all three cytosine nucleosides in the guide strand and all seven cytosine nucleosides in the passenger strand are replaced with ara-C. In another specific composition, all three cytosine nucleosides in the guide strand and all seven cytosine nucleosides in the passenger strand are replaced with ara-C and all seven uracil bases in the guide strand are replaced with 5-FU.

Any of the above-described miRNA mimic compositions can be used in the method for killing a cancer cell set forth in the SUMMARY section. In this method, the miRNA mimic is miR-15a, miR-16, miR-129, miR-194, miR-192, miR-139, miR-140, or miR-145 in which the guide strand contains both ara-C and 5-FU and the passenger strand contains ara-C.

The miRNA mimic compositions can also be used in the method for treating cancer disclosed above. The method features administering the miRNA mimics described above to a subject suffering from cancer. The cancers that can be treated include, but are not limited to, leukemia, lymphoma, or multiple myeloma. For example, acute myeloid leukemia (“AML”) or acute lymphocytic leukemia (“ALL”) can be treated with the disclosed method.

Administration routes of the miRNA mimic compositions include, but are not limited to oral administration, parenteral administration (e.g., subcutaneous, intramuscular, intraperitoneal, or intravenous injection), and topical administration. Additional routes that can be used are set forth in Ju et al.

In the cancer-treating method, the miRNA mimics can be miR-15a, miR-16, miR-129, miR-194, miR-192, miR-139, miR-140, and miR-145 in which the guide strand contains both ara-C and 5-FU or solely 5-FU and the passenger strand contains ara-C.

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety.

### EXAMPLES

### Example 1: Preparation of miRNA Mimic of miR-15a

The modified miR-15a mimic was synthesized with standard phosphoramidite chemistry as two separate RNA oligonucleotides, i.e., guide strand and passenger strand. The uracil bases were replaced with 5-FU by including in the reaction a 5-fluorouracil nucleoside phosphoramidite as a precursor, along with the phosphoramidite derivatives of nucleosides containing natural bases, e.g., A, G, and C). Similarly, an ara-C phosphoramidite was included during synthesis with natural A, U, and G to produce ara-C substituted miRNA molecules.

Different combinations of unmodified and modified guide and passenger strands were produced as shown in FIGS. 1A-1E. An miR-15a mimic was produced in which the passenger strand was not modified and the guide strand was modified by replacing the three cytosine nucleosides in the guide strand with ara-C. See FIG. 1A. Additional modified miR-15a mimics were made including unmodified passenger strand paired with and ara-C modified guide strand (see FIG. 1B), unmodified passenger strand paired with 5-FU modified guide strand (see FIG. 1F), ara-C modified passenger strand paired with unmodified guide strand (see FIG. 1C), and ara-C modified passenger strand paired with 5-FU modified guide strand (see FIG. 1D).

Purified passenger and guide strands were annealed before use in transfection assays. Briefly, the passenger and guide strands were each dissolved in 100 mM acetic acid pH 3.8 to a concentration of 100 μM and mixed at a 1:1 volume ratio. The mixture was heated at 60° C. for 45 min. and then cooled at room temperature for 30 min. The annealed RNA was desalted by ethanol precipitation with 3M sodium acetate.

### Example 2: Cell Viability of Cancer Cells Exposed to Modified miRNA Mimics

### Cell Lines and Culture

MV-4-11 (American Type Culture Collection; “ATCC”) AML cells were maintained in Iscove's Modified Dulbecco's Medium (IMDM) (Thermo Fisher Scientific) supplemented with 10% fetal bovine serum (“FBS”; VWR). REH ALL cells (ATCC) were maintained in RPMI-1640 Medium (Thermo Fisher Scientific) supplemented with 10% FBS.

### Transfection

For transfection with a vehicle, twenty-four hours before transfection, cells were plated in 6 well plates at 1×10⁵ cells per well. Cells were transfected with negative control miRNA (Thermo Fisher Scientific), 5-FU miR-15a, or various versions of the ara-C modified miR-15a mimic, using Lipofectamine™ 2000 (Thermo Fisher Scientific) as directed by the manufacturer. MV-4-11 cells were transfected with 15 nM of miRNA and REH cells were transfected with 25 nM of miRNA. Twenty-four hours post-transfection, cells were replated at 2000 cells per well in 96 well plates.

For vehicle free transfection, cells were plated in 96 well plates at 2000 cells per well. Twenty-four hours later, miRNA was diluted in cell media and added to the 96 well plates. MV-4-11 cells were treated with 5 nM, 2.5 nM, and 1.25 nM miRNA. REH cells were treated with 10 nM, 5 nM, and 0.5 nM miRNA.

### Cell Viability

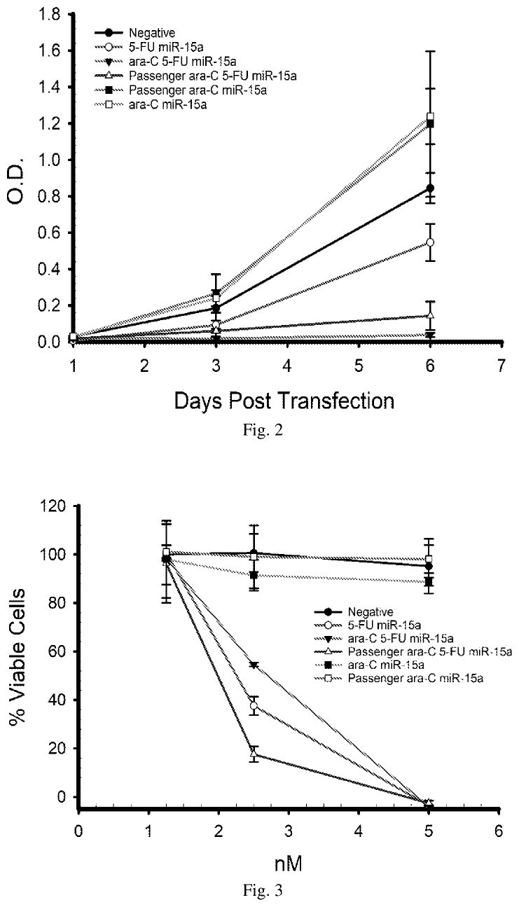

Cell numbers were measured on days 1, 3, and 6 post transfection for cells transfected with Lipofectamine 2000 and on day 6 post transfection for vehicle free transfection, using WST-1 dye (Roche). Briefly, cells were incubated with 10 μl of WST-1 dye per 100 μl of media for 1 hour (REH) or 2 hours (MV-4-11) at 37° C. and absorbance was read at 450 nm and 630 nm. The optical density (“OD”) was calculated by subtracting the absorbance at 630 nm from that at 450 nm. OD correlates with cell viability. In certain instances, OD was converted to percent viable cell relative to the negative control. The results are shown in FIGS. 2-5.

In MV-4-11 AML cells subjected to lipofection, both ara-C 5-FU miR-15a and passenger ara-C 5-FU miR-15a were more effective at inhibiting cell viability, as compared to both 5-FU miR-15a and ara-C miR-15a. See FIG. 2.

When MV-4-11 cells were transfected vehicle free, only passenger ara-C 5-FU miR-15a was better than 5-FU miR-15a at inhibiting cell viability. See FIG. 3. 5-FU miR-15a, ara-C 5-FU miR-15a and passenger ara-C 5-FU miR-15a each killed all cells in 6 days at a dosage of 5 nM. See id.

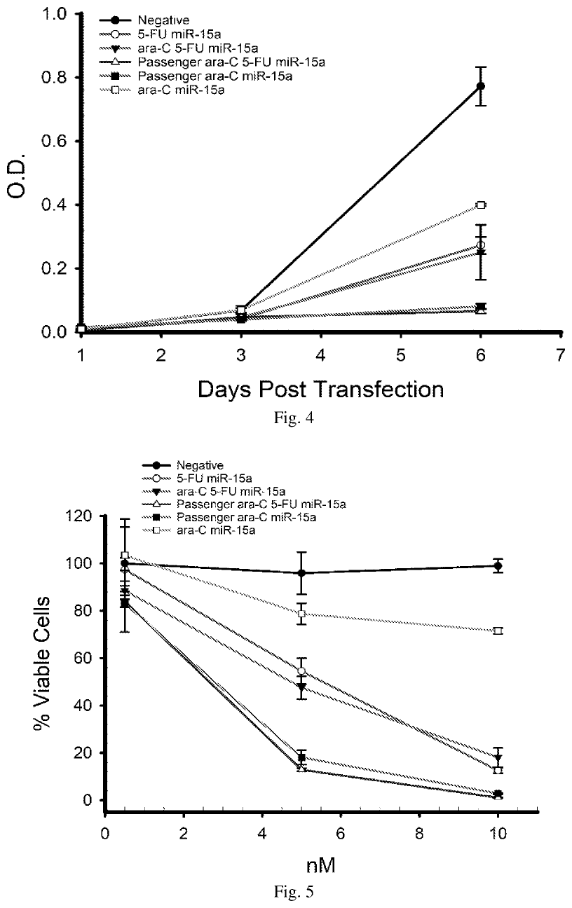

The viability of REH ALL cells was decreased by all modified miRNAs introduced by lipofection, as compared to control miRNA. See FIG. 4. In particular, passenger ara-C 5-FU miR-15a and passenger ara-C miR-15a were both more effective than 5-FU miR-15a at inhibiting cell viability. See id. Ara-C 5-FU miR-15a behaved similarly to 5-FU miR-15a in this assay, and ara-C miR-15a was slightly less effective than 5-FU miR-15a. See id.

When REH cells were transfected without a vehicle, again all modified miRNAs tested inhibited cell viability. See FIG. 5. As in lipofected REH cells, passenger ara-C miR-15a and passenger ara-C miR-15a were most effective at inhibiting cell viability. See id. In addition, ara-C 5-FU miR-15a reduced cell viability to a degree similar to 5-FU miR-15a and were both more effective than ara-C miR-15a. See again FIG. 5.

### Example 3: Inhibition of Wee1 Expression by Modified miR-15a

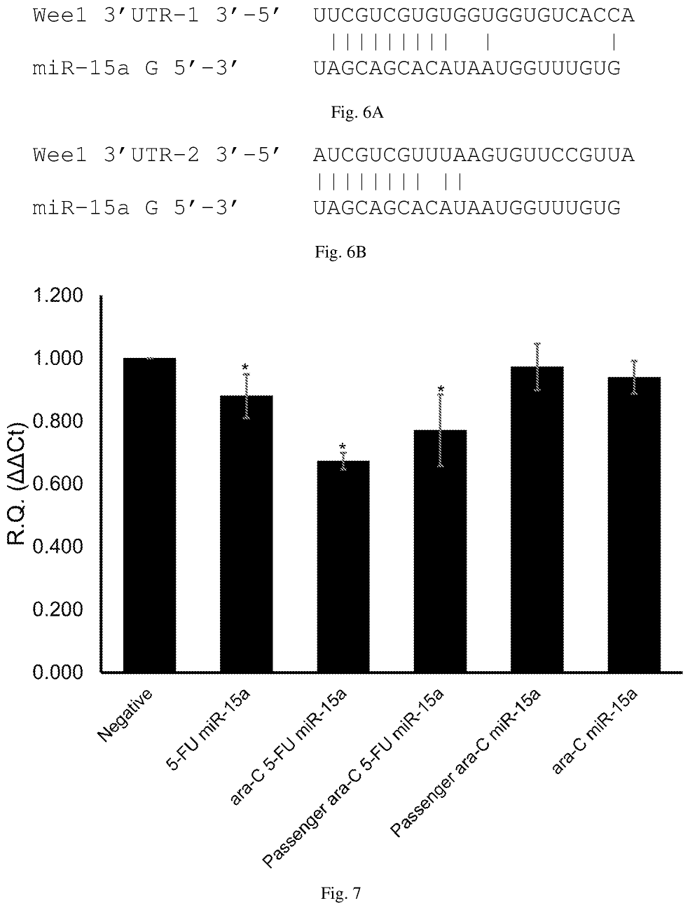

Wee1 is an important cell cycle regulator and a potential target for AML therapy. Wee1 is a direct target of miR-15a with two miR-15a binding sites in the 3′ UTR of Wee1. See FIGS. 6A and 6B.

### Transfection

For transfection with a vehicle, twenty-four hours before transfection, cells were plated in 6 well plates at 1×10⁵ cells per well. Cells were transfected with negative control miRNA (Thermo Fisher Scientific), 5-FU miR-15a, or various versions of the ara-C modified miR-15a mimic, using Lipofectamine™ 2000 (Thermo Fisher Scientific) as directed by the manufacturer. MV-4-11 cells were transfected with 15 nM of miRNA. Twenty-four hours post-transfection, RNA was isolated from cells using TRIzol™ (Thermo Fisher Scientific) reagent following the manufacturer's protocol. Wee1 expression was measured using qRT-PCR and normalized to GAPDH expression and calculated by the double delta threshold cycle (ΔΔCt) method. The results are shown in FIG. 7.

miR-15a modified only with 5-FU on the guide strand was able to significantly suppress Wee1 gene mRNA expression, yet, miR-15a modified with ara-C alone on the guide strand did not. Compare 5-FU miR-15a to ara-C miR-15a in FIG. 7. miR-15a modified on the guide strand with both ara-C and 5-FU (ara-C 5-FU miR-15a in FIG. 7) was able to significantly inhibit expression of Wee1. Clearly, the combination of these modifications improves the ability of miR-15a to inhibit expression of Wee1.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. A composition comprising a micro-RNA mimic that contains one or more cytosine nucleosides in which at least one cytosine nucleoside is replaced by cytosine arabinoside.

2. The composition of claim 1, wherein the micro-RNA mimic includes a guide strand and a passenger strand.

3. The composition of claim 2, wherein the cytosine arabinoside is present in the passenger strand.

4. The composition of claim 2, wherein the cytosine arabinoside is present in the guide strand.

5. The composition of claim 3, wherein the cytosine arabinoside is present in the guide strand.

6. The composition of claim 5, wherein all of the one or more cytosine nucleosides in the passenger strand are replaced with cytosine arabinoside.

7. The composition of claim 6, wherein all of the one or more cytosine nucleosides in the guide strand are replaced with cytosine arabinoside.

8. The composition of claim 2, wherein the guide strand contains one or more uracil bases in which at least one uracil base is replaced by 5-halouracil.

9. The composition of claim 8, wherein the 5-halouracil is 5-fluorouracil.

10. The composition of claim 8, wherein all of the one or more uracil bases are replaced with 5-fluorouracil.

11. The composition of claim 10, wherein the guide strand or the passenger strand contains one or more cytosine nucleosides in which all of the one or more cytosine nucleosides are replaced by cytosine arabinoside.

12. The composition of claim 11, wherein the micro-RNA mimic is selected from the group consisting of miR-15a, miR-16, miR-129, miR-194, miR-192, miR-139, miR-140, and miR-145.

13. The composition of claim 12, wherein the guide strand has the sequence of UAGCAGCACAUAAUGGUUUGUG (SEQ ID NO: 1) and the passenger strand has the sequence of CAGGCCAUAUUGUGCUGCCUCA (SEQ ID NO: 2).

14. A method for killing a cancer cell, comprising contacting the cancer cell with the composition of claim 12.

15. A method for treating cancer, the method comprising identifying a subject suffering from cancer and administering to the subject an effective amount of a composition containing a micro-RNA mimic having a guide strand and a passenger strand that each contains one or more cytosine nucleosides in which at least one cytosine nucleoside in the guide strand or at least one cytosine nucleoside in the passenger strand is replaced by cytosine arabinoside.

16. The method of claim 15, wherein the cancer is leukemia, lymphoma, or multiple myeloma.

17. The method of claim 16, wherein all of the one or more cytosine nucleosides in the guide strand or in the passenger strand are replaced with cytosine arabinoside.

18. The method of claim 17, wherein the guide strand contains one or more uracil bases in which at least one uracil base is replaced with 5-fluorouracil.

19. The method of claim 18, wherein all of the one or more uracil bases are replaced with 5-fluorouracil.

20. The method of claim 19, wherein the micro-RNA mimic is selected from the group consisting of miR-15a, miR-16, miR-129, miR-194, miR-192, miR-139, miR-140, and miR-145.
